(19) United States
(12) Patent Application Publication
(10) Pub. No.: US 2007/0276393 A1
(43) Pub. Date: Nov. 29, 2007

(54) Surgical Reamer and Blades for Said Reamer

(76) Inventor: Lucia Bonadei, Corcelles (CH)

Correspondence Address: BACON & THOMAS, PLLC, 625 SLATERS LANE, FOURTH FLOOR, ALEXANDRIA, VA, 22314, US

(21) Appl. No.: 10/580,277

(22) PCT Filed: Nov. 29, 2004
(86) PCT No.: PCT/CH04/00716

§ 371 Date: Mar. 26, 2007

(30) Foreign Application Priority Data
Nov. 28, 2003 (CH) 2025/2003

(51) Int. Cl. A61B 17/58

(52) U.S. Cl. 606/80

(57) ABSTRACT

The acetabular cup bur comprises four blades (1, 2, 3, 4) which are preferably stamped out of non-hard metal sheets, e.g. stainless steel. The blades have slits (6, 7, 8, 9, 10) which enable them to be inserted into each other in order to obtain a bur head. The slits have different widths, enabling more than two of them to be assembled on the axis of rotation. Each blade is extended by a lower part (19). Said lower parts are driven by a flat ring (15) which keeps them rigid. The blades are stamped. The cutting wire (18) of the blade is obtained by the same stamping operation due to an appropriate form of the stamps.

### TECHNICAL FIELD

This invention relates to a surgical reamer and, more specifically, to an acetabular reamer, intended for reaming the hip bone for the purpose of inserting a prosthesis.

### BACKGROUND ART

Known reamers, for use in medical applications, and particularly acetabular reamers, are shaped like a hemispherical cap, with openings and cutting edges, similar to those of cheese graters; such reamers are formed by stamping, blanking and spinning.

Examples of such hemispherical reamers may be found in documents CH 692600, CH 690021, FR 2281095, U.S. Pat. No. 4,811,632, and U.S. Pat. No. 5,100,267.

Reamers of the type mentioned above are relatively expensive to manufacture, mainly because of the various operations involved in this kind of manufacture.

In the medical field, particularly that of acetabular reamers, the cost of reamers prevents them from being used only once, whereas single usage would be far preferable because of septic risks.

The document U.S. Pat. No. 5,100,267 describes a disposable acetabular reamer cup. In order to reduce its price, the acetabular reamer described in that document is fitted with a polymeric plug, which is easy to make and inexpensive. However, the cutting bowl, in stainless steel, must be manufactured by conventional means. This means that it remains expensive. Moreover, the presence of plastic matter often presents risks, since this type of very soft material can easily leave particles in the body that are often not well tolerated by the patient.

Consequently, medical practitioners continue to use acetabular reamers, usually free of any plastic material, that are expensive and that they must sterilise after each use, which involves relatively substantial costs.

Moreover, reamers are of solid construction, such that the reamer has no interstitial space that allows the user to view the bottom of the cavity hollowed out by the reamer. The user is therefore hardly able to check the progress of the reaming during the operation, or the state of the reamed cavity walls. This is a serious inconvenience, particularly in the medical field.

Other reamers have cutting blades which are radially disposed, equiangularly spaced around the axis of rotation, and which extend outwards. Such examples may be found in patents FRI041311, FRI031888, U.S. Pat. No. 3,702,611 and U.S. Pat. No. 4,621,637. In these examples, the blades are built-up, i.e. manufactured independently and then fixed in the cutter head, which contains housings for this purpose. Means of attachment are required (such as screws or plugs), which complicate the device.

This type of reamer has the same inconveniences as mentioned above, and particularly the difficulty experienced by the surgeon in viewing the bottom of the cavity cut by the reamer. Moreover, such reamers are still expensive to manufacture.

PCT application No. W098/31291 shows a relatively simple acetabular reamer, in which flat blades are stamped in a semi-circular shape from sheet metal and are assembled together by means of slots in the blades themselves. The blades cross each other at right angles and are semi-circular. To attach them on to the tool and hold them together, the blades are inserted by means of projections at both ends of each blade into apertures in a base plate. This plate has a central aperture which allows it to be fixed on to a rotating tool.

This reamer model still has the inconvenience of reduced visibility, due to the presence of the base plate. There is also the question of whether the reamer is sufficiently rigid.

PCT application No. WO02/49516 shows another surgical reamer formed from blades stamped from sheet metal and fitted together by means of slots. There are only two blades, which join at the axis of rotation, the mounting slots also being on this axis.

In this reamer, visibility is much better, the surgeon being able to see through the blades to the bottom of the cavity cut by the reamer.

The reamer in question, however, is not very rigid, despite the depositor's claim. Moreover, the efficiency is probably affected by the fact that there are only two blades.

The present invention intends to provide a reamer composed of blades assembled together by means of slots wherein the longitudinal axis of symmetry is coincident with the axis of rotation, there being at least four such blades. Such a reamer, which is easy to manufacture, will be more rigid and will cut more efficiently than known reamers, while guaranteeing optimum visibility during the operation. Moreover, it is less difficult to manufacture, since the blades are all stamped from metal sheets.

The invention also intends to provide blades that would allow such a construction.

### DISCLOSURE OF THE INVENTION

Generally, the surgical reamer which is the subject of the invention consists of several fiat blades assembled together by means of slots wherein the longitudinal axis of symmetry is coincident with the axis of rotation of the reamer; the reamer comprises at least four blades; or one or several slots of said blades, or one or several sectors of said slots, are of a different width from the other slots and/or sections of slots.

In the preferred embodiment of the invention, the reamer comprises four blades, the first of which has at least one slot which runs from the lower edge of said blade; the second blade has at least one slot running from the top of the blade, and each of said slots has, at least along a section of its length, a width corresponding to the thickness of the blade containing the other slot, the third blade has at least one upper slot running from the top of the blade, and at least one lower slot running from the lower edge of the blade the width of the upper slot being wider than the width of the lower slot; the fourth blade has at least one slot running from the top of the blade, this slot comprises an outer section and an inner section, the outer section being wider than the inner section; moreover, the width of the lower slot of the third blade corresponds to the thickness of the fourth blade; the width of the inner section of the slot of the fourth blade corresponds to the thickness of the third blade, the width of the upper slot of the third blade and the width of the outer section of the slot of the fourth blade are the same and are sized such that once fitted together, the two first blades can be inserted into said upper slot of the third blade and into the outer section of the fourth blade, the third and fourth blades being themselves fitted together.

In a second embodiment, in which it is easier to obtain blades of equal height (following the axis of rotation of the reamer), a form of embodiment which is applicable both to the general embodiment and to the first exemplary and preferred embodiment described above, the slot of the first cutting blade has two sections of different widths, i.e. an outer sector, close to the lower edge of the blade, larger than the inner section, which is closer to the top of the blade.

In a third embodiment, which has the same object as the second, the second cutting blade has at least one lower slot running from its lower edge, and this lower slot is wider than the slot that runs from the top of the blade.

In a fourth embodiment, applicable to the preceding ones, the reamer comprises at least one rigidifying element which partially encircles and holds together the cutting blades making up the reamer.

In a fifth embodiment, applicable to the preceding embodiments, the rigidifying element is a cylindrical bushing.

In a sixth embodiment, applicable to the preceding embodiments, and which is the preferred embodiment, the rigidifying element is a flat ring containing notches in which the cutting blades are inserted, the plane of said ring being perpendicular to the axis of rotation of the reamer.

Each blade of the reamer preferably comprises at least one slot wherein the longitudinal axis of symmetry is coincident with the axis of rotation of the reamer, one rounded cutting part, preferably of semi-circular shape, and one lower part that is narrower than the cutting part.

In a exemplary embodiment, for use in a reamer described above wherein the blades may more easily be of equal height (second embodiment), at least one blade comprises one or several slots at least one of which has sections of different widths.

In a exemplary embodiment of such a blade, it comprises a slot running from the top of the cutting part and a lower slot running from the lower edge, the width of the upper slot being different from the width of the lower slot.

In an even more exemplary embodiment of such a blade, said upper or lower slot comprises at least one outer section and one inner section, the width of the outer section being greater than the width of the inner section.

### BRIEF DESCRIPTION OF THE DRAWINGS

FIG. 1 shows a cavalier projection of an exploded view of a reamer according to the invention in which the reamer contains four blades.

FIG. 2 is a cavalier projection of a perspective view of a reamer in the same embodiment as that of FIG. 1, the elements being assembled to form the reamer.

FIG. 3 is a longitudinal section of a reamer in the embodiment in FIG. 2.

FIG. 4 is a top view of a reamer in the embodiment in FIGS. 2 and 3.

FIG. 5 is a cavalier projection of a reamer in a slightly different embodiment than that of FIGS. 1 to 4, in which a shorter bushing provides the rigidity for the blade assembly.

FIG. 6 is a longitudinal section of a reamer according to the invention in an exemplary embodiment, in which a second bushing provides the rigidity for the blade assembly.

FIG. 7 is an enlarged view of the centre of FIG. 4, showing the intersection of the blades and the relative dimensions of the slots cut in the blades for their insertion into one another.

FIG. 8 is a front view of a first blade for a reamer according to the to the invention, in the second embodiment.

FIG. 9 is a front view of a second blade for a reamer according to the invention, in the third embodiment.

FIG. 10 is a front view of a third blade for a reamer according to the invention, for use in the other embodiments mentioned and particularly in accordance with the ninth.

FIG. 11 is a front view of a fourth blade for a reamer according to the invention, for use in the other embodiments mentioned and particularly in accordance with the second.

FIG. 12 is a front view of a ring for a reamer according to the invention, intended to ensure the rigidity of the reamer once assembled, in accordance with the sixth embodiment.

FIG. 13 is a cavalier projection of a reamer resulting from the blade assembly represented in FIGS. 8 to 11 and of the flat ring shown in FIG. 12.

### BEST MODE FOR CARRYING OUT THE INVENTION

An important characteristic of the invention is the fact that the blades may be cut from sheet metal. The term “sheet metal” does not include hard, carbide type, metal sheet, but simply sheet metal that can be stamped, such as stainless steel. Blades 1, 2, 3 and 4 of the reamer according to the invention are cut from sheet metal; in the preferred form of the invention, the blades are stamped. This method has the advantage of being very low-cost. Laser cutting or spark erosion machining is also possible, but at a less advantageous price.

Preferably, the same operation that stamps the blade will also give it a sharp cutting edge 18. It is obviously possible, however, to obtain a cutting-edge by means of a conventional grinding operation.

Likewise, and preferably, the teeth 16 around the edge of the blade as well as the possible angle of said blades in relation to the plane of the blade, may be obtained by stamping, in a single operation.

Experience shows, however, that such an angle is not essential.

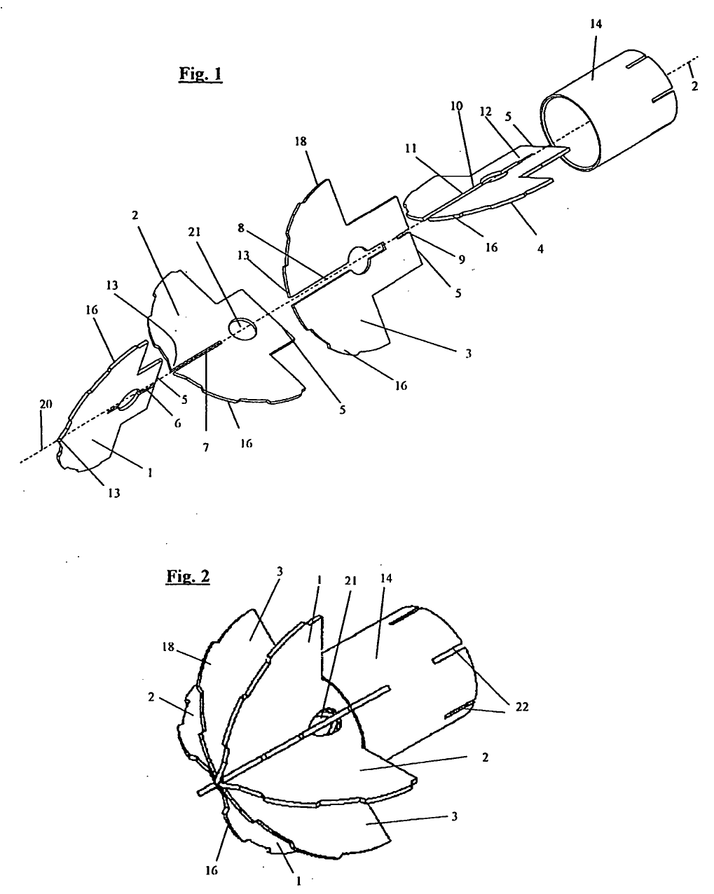

In the preferred embodiment of the invention, the reamer is made up of four blades 1, 2, 3 and 4. Each blade has a longitudinal axis of symmetry coincident with the axis of region of the reamer 20. The blade edge, i.e. the cutting edge 18, has a semi-circular shape here. This shape is particularly well adapted to acetabular reamers. It is obvious, however, that numerous other shapes may be used, depending on the intended purposes of the reamer. Each blade has at least one slot 6, 7, 8, 9, or 10, which is along the axis of rotation 20. This longitudinal slot allows the blades to be inserted into one another. Each blade also had a lower part 19, which here has the shape of a rectangle or a double rectangle which extends the blade in the direction of the rotary drive shaft of a tool, not represented here, on to which the reamer must be fixed. It is this lower part 19 which enables this assembly. It may be seen in FIG. 1 that the slot 6 in the first blade 1 runs from the edge 5 of the lower part 19 to halfway up the blade. By contrast, the slot 7 in the second blade 2 runs from halfway up the blade to the top 13 of the blade. In this way, the first two blades fit into each other at right angles. With respect to the following two blades, they have slots 8, 9 and 10 of a more complex shape and disposition. It may be noted first that, in FIG. 1, the lower parts 19 of these second two blades are longer than those of the first two. Indeed, the third and fourth blades each have a slot, 8 and 10 respectively, which runs from their top 13 to their lower part 19. Moreover, the slots 8 and 10 of the third and fourth blades are wider than those of the first two blades. The third blade 3 also has a lower slot 9 which runs from its lower edge 5 to a quarter of the way up said lower part; this lower slot 9 is narrower than the upper slot 8. The slot 10 in the fourth blade has two sections 11 and 12; the outer section 11, which runs from the top 13 of the fourth blade to halfway up the lower part 19, is wider than the inner section 12. The width of the lower slot 9 of the third blade is the same as that of the inner section 12 of slot 10 in the fourth blade and corresponds to the thickness of the blade inserted into it. The length of slot 8 in the third blade and that of the outer section 11 of the fourth blade is the same and corresponds to the total height of each of the first two blades. The third blade 3 and the fourth blade 4 are joined to each other by the lower slot 9 and by the inner section 12, at right angles.

The group formed by the two blades 1 and 2 is then inserted into the upper slot 8 and into section 11 of the group formed by the third and fourth blades. The need for the greater width in the upper slot 8 and in the outer section 11 for insertion of the first two blades is shown in FIGS. 4 and 7. FIG. 7 shows that the width of slots 6 and 7 must correspond to thickness “a” for both blades 1 and 2 which cross each other in an “X” shape in this drawing. The two blades which cross each other in a <<<+>> shape (and which correspond to the two blades 3 and 4), have an upper slot 8 and a section 11 with a width “b”, which allows the insertion of the group formed by the two left blades. It can be seen here that width “b” is considerably greater than width “a” (according to Pythagoras, b=a(1+V2)). An accurate adjustment of the widths a and b of the slots and blades will prevent any movement of the blades against each other and achieve a rigid assembly.

Once the four blades are slotted together, their lower parts 19 are, for example, driven into a bushing 14, which is concentric to the axis of rotation 20 of the reamer. Thus fixed on the periphery of their lower parts, the blades form a very rigid assembly. The presence of such a bushing 14, however, is not always essential: in several applications of the reamer, there is already sufficient rigidity if the widths a and b of the slots are correctly adjusted.

The blade assembly can also be welded or glued into the bushing, or fixed by any other means. Driving them in remains the simplest method. Likewise, the bushing may be of some other shape than a cylinder, the cylindrical shape remaining the most rational.

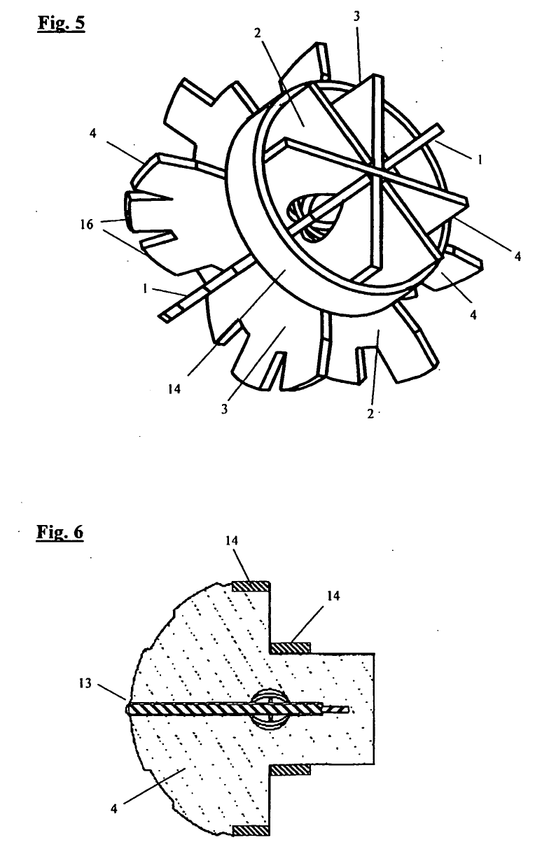

This assembly may be made even more rigid, if necessary, by adding a second bushing 14, of a greater diameter, which encircles the blades at a greater distance from the axis of rotation 20. This form of exemplary embodiment is shown in FIG. 6. It is also possible only to place the bushing 14 on the periphery of the reamer, without another bushing to hold the assembly around the lower parts 19 of the blades.

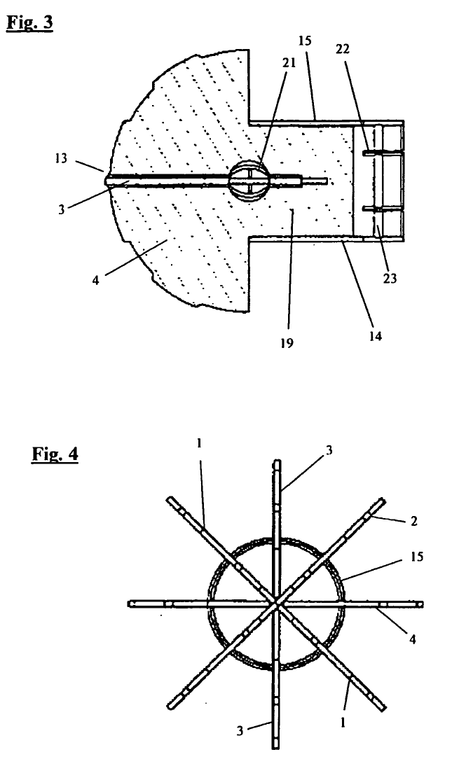

In an embodiment represented in FIGS. 1 to 3, the bushing 14 not only serves to fix the blades 1 solidly together, but also to longitudinally fix the reamer, i.e. in the direction of the axis of rotation 20, on to the rotary drive shaft which moves it. The bushing 10 thus contains, in the part opposite to that which houses the lower parts 19 of the blades, several longitudinal slots 22. These slots 22 render the bottom of the bushing more flexible and allow the bushing to lock easily on to a rotary drive shaft and to be fixed there thanks to the notch 23 shown in FIG. 3. However, this method of fixation is far from being the only one possible.

Another method of making the blade assembly rigid is to replace the bushing by a plate, preferably in the form of a flat ring 15, which has notches 17 in which the blades are inserted. FIG. 12 shows such a ring, and FIG. 13 shows it mounted around the lower parts 19 of the blade assemblies.

The axis of symmetry of the blades 1 is the axis of rotation 20 of the reamer. In order that the blades may be sharpened, if necessary, it is advisable to provide for a hole 21 to be placed on this axis of rotation.

In the drawings, the blades have a semi-circular cutting edge 18 with teeth 16, which can be used to facilitate reaming depending on the intended purpose. As was seen above, preferably, and as far as possible, the teeth are cut and their angle is obtained in one and the same stamping operation.

It was seen, in the embodiment represented in FIGS. 1 to 3, that the height of the first two blades 1 and 2 was less than that of the third and fourth blades 3 and 4. However, it could be advantageous to have an equal height on each blade, particularly with a view to mounting the reamer on a tool.

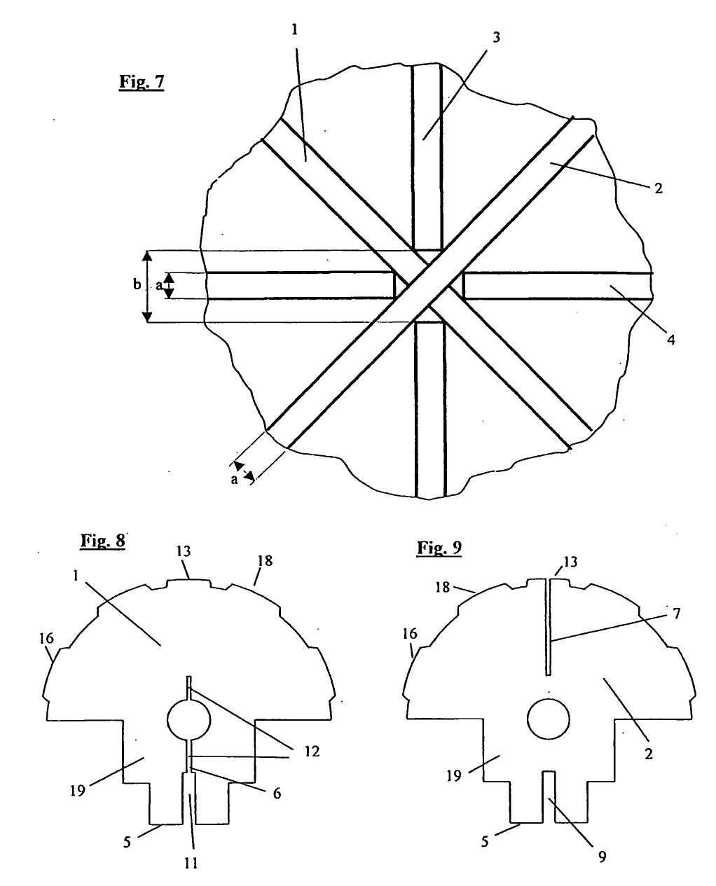

In a preferred embodiment of the invention, the first blade 1, as represented in FIG. 8, has a slot 6 comprising two sections: an outer section 11, which runs from the lower edge 5 of the blade, and an inner section 12, which prolongs the outer section in the direction of the top 13, and which is narrower than the outer section. Likewise, as shown in FIG. 9, the second blade 2, in addition to the upper slot 7, which runs from the top 13 to the bottom, has a lower slot 9 which runs from the lower edge 5 towards the top.

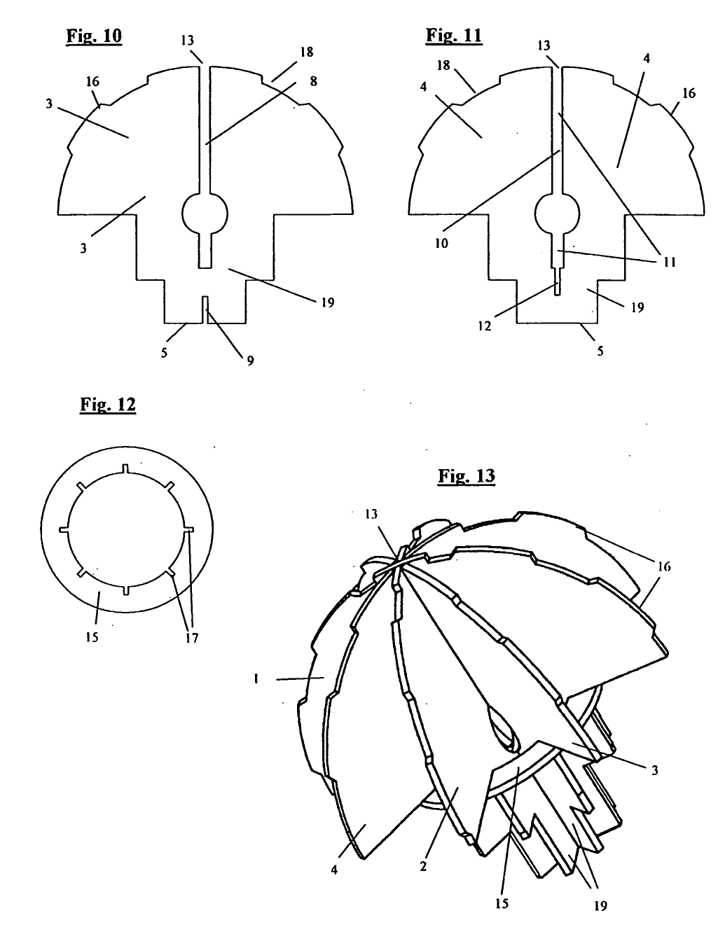

The outer section 11 of the first blade 1 and the lower slot 9 of the second blade 2 are wider than the inner sector 12 and the upper slot 7 respectively. This width allows lower part 9 to be lengthened, to obtain the same height as in the third and fourth blades 3 and 4, represented in FIGS. 10 and 11. The increased width of outer sector 12 and inner slot 9 allows sufficient place to house the assembly formed by the third and fourth blade assembly.

The thinness of the stamped sheet metal blades, and their position in the plane of the axis of rotation, allows the user to see the part to be reamed, through the blades, even during the operation.

The presence of four blades instead of only two ensures sufficient rigidity to perform operations without any greater risk than with a conventional reamer.

Manufacturing the blades, and their assembly, is easy and inexpensive; they may be disposed of instead of cleaned, an onerous operation.

### INDUSTRIAL APPLICABILITY

The reamer and blades of this invention are essentially for use in operations for fitting hip prostheses.

1-11. (canceled)

12. Surgical reamer comprising at least four flat blades assembled together by means of slots in said blades, said reamer having a longitudinal axis of symmetry that is coincident with an axis of rotation of the reamer, and further wherein at least one of the slots and/or sections of said slot or slots has a different width from that of at least one other slot and/or section of a slot.

13. Reamer according to claim 12, wherein the first of said four blades has at least one first slot which runs from the lower edge of the blade, the second of said four blades having at least one second slot running from the top of the blade, each of said first and second slots having, at least in one section, a width corresponding to the thickness of the blade which is fitted into the other slot, the third blade of said four blades has at least one upper third slot running from the top of said third blade and at least one lower third slot running from the lower edge of said third blade, the width of said upper slot being greater than the width of said lower slot, and the fourth blade of said four blades has at least one fourth slot running from the top of said blade, said fourth slot comprising at least one outer section and one inner section, the width of said outer section being greater than the width of said inner section, the width of the lower third slot of the third blade corresponding to the thickness of the fourth blade, the width of the inner section of the fourth slot of the fourth blade corresponding to the thickness of the third blade, and the width of the upper third slot of the third blade and the outer section of the fourth blade being the same and such that, once fitted together, the first two blades can be inserted into said upper third slot of the third blade and into the outer section of the fourth blade, said third and fourth blades being themselves fitted together.

14. Reamer according to claim 12, wherein the first slot of a first one of said blades has at least an outer and an inner section, the outer section being wider than the inner section.

15. Reamer according to claim 12, wherein a second one of said blades has at least one lower slot running from its lower edge, said lower slot being wider than the second slot.

16. Reamer according to claim 12, comprising at least one rigidifying element adapted to encircle and hold together the four blades.

17. Reamer according to claim 16, wherein the rigidifying element comprises a cylindrical bushing.

18. Reamer according to claim 16, wherein the rigidifying element comprises a flat ring with notches into which the blades are inserted.

19. Blade for a reamer according to claim 12, comprising at least one slot having sections of different widths.

20. Blade according to claim 19, wherein said at least one slot comprises an upper slot running from the top of a cutting part of the blade and a lower slot running from the lower edge of the blade, the width of the upper slot being different from the width of the lower slot.

21. Blade according to claim 20, wherein one of said upper or lower slots comprises at least one outer section and one inner section, the width of the outer section being greater than the width of the inner section.

22. Blade according to claim 19, comprising at least one slot wherein the longitudinal axis of symmetry is coincident with the axis of rotation of the reamer, a rounded cutting part, and a lower part which is narrower than the cutting part.
